(19) United States
(12) Patent Application Publication
(10) Pub. No.: US 2019/0326003 A1
(43) Pub. Date: Oct. 24, 2019

(54) METHOD OF MANAGING PRESCRIPTION MEDICAL AND PHARMACEUTICAL PRODUCTS AND MEDICAL BENEFITS

(71) Applicant: BenMedica, LLC, St. Louis, MO (US)

(72) Inventors: Bruce Scripps Wilkinson, St. Louis, MO (US); David Bradsher, St. Louis, MO (US)

(21) Appl. No.: 16/393,447

(22) Filed: Apr. 24, 2019

Related U.S. Application Data
(60) Provisional application No. 62/661,956, filed on Apr. 24, 2018.

(51) Int. Cl. G16H 20/10; G16H 10/60; G06Q 30/06; G06Q 30/02; G06F 16/2457; G06F 16/248; G06F 16/9035

(52) U.S. Cl. CPC G16H 20/10; G16H 10/60; G06Q 30/0629; G06F 16/9035; G06Q 30/0641; G06F 16/24578; G06F 16/248; G06Q 30/0283

(57) ABSTRACT

A method of processing drug alternatives for a selected drug. The method includes receiving a selection of the selected drug and generating a drug alternatives list from source data, the drug alternative list including a plurality of drug alternatives. The method includes gaining access to a price model and at least one price data source. The method includes determining respective prices for the plurality of drug alternatives according to the price model and the at least one price data source and ranking the plurality of drug alternatives in the drug alternatives list according to respective ordinality and respective prices. The method includes suppressing ones of the plurality of drug alternatives lacking a desired clinical outcome from the drug alternatives list. The method includes filtering resultant drug alternatives by at least one parameter. The method includes transmitting the drug alternatives list to a user computing system for display.

### CROSS-REFERENCE TO RELATED APPLICATIONS

The application claims priority to U.S. Provisional Patent Application Ser. No. 62/661,956 filed on Apr. 24, 2018 titled “Method of Managing Prescription Medical and Pharmaceutical Products and Pharmacy Benefits,” the entire contents of which are hereby incorporated herein by reference.

### FIELD

The field of the disclosure relates generally to the process of processing medical and pharmaceutical products and, more specifically, a system and method for enhancing, configuring, and viewing formulary and benefit data, real-time insurance benefit verification check data, and other custom data sets available to payers, providers, patients, and other users.

### BACKGROUND

Prescription pharmaceutical products and services, e.g., drugs, and medical devices such as syringes and lancets move in a unique market defined by manufacturers, wholesalers, pharmacies, and patients. The market and, in particular, the selection of drugs is largely defined by physicians, or prescribers, and payers as drugs move from manufacturers to wholesalers, from wholesalers to pharmacies, and from pharmacies to patients. Payers generally negotiate drug prices and patient copay amounts. Physicians generally make drug selection choices based on formulary status, coverage restrictions, patient copay, and increasingly, the anticipated total cost of the drug.

Drugs are first sold by manufacturers to wholesalers, who often receive discounts and rebates for the quantities purchased. These transactions are the basis for an average manufacturer price (AMP), a wholesale acquisition cost (WAC), and an average sales price (ASP). AMP is a measure of the wholesaler's cost of a drug directly from the manufacturer after rebates and discounts. WAC is a measure of a manufacturer's list price to wholesalers or direct purchasers before discounts and rebates. ASP is also a measure of cost to wholesalers or direct purchasers, but generally with discounts and rebates, and also only for drugs covered by Medicare Part B.

Drugs are typically then sold by wholesalers to pharmacies. These transactions are the basis for an average wholesale price (AWP), an estimated acquisition cost (EAC), and an average actual cost (AAC). The AWP is a measure of price paid by pharmacies to purchase a given drug, which can often be found in the drug compendia, e.g., Medi-Span or First Databank. EAC is a measure of price paid by state Medicaid programs and generally reflects the Medicaid reimbursement amount plus a dispensing fee for a given drug. AAC is a measure of price paid by pharmacies to wholesalers after discounts.

Drugs are then sold to the patients, i.e., consumers, at a “usual and customary” (U&C) price, which is also referred to as a cash price, i.e., without insurance. Most patients are enrolled with a third-party payer, e.g., insurance, that manages prescription medical goods and services using a pharmacy benefits manager (PBM), which is often another third party. Accordingly, the term “payer” may include a health insurance plan or any entity acting on their behalf, such as a PBM or third party administrator (TPA). The patients may pay, for example, a copay at the point of sale, i.e., the pharmacy, and the payer reimburses the pharmacy for the balance of the cost. Generally, the cost of a given generic drug at the point of sale is the federal upper limit (FUL) or the maximum allowable cost (MAC). Certain branded drugs have respective negotiated prices with payers.

A typical sale by the pharmacy to a patient begins with a physician's prescribing a drug for a patient. When writing the prescription for a given drug, the physician typically sees formulary and benefit (F&B) information. The physician generally knows efficacy information for a drug and possibly some limited information on appropriate drug alternatives. If drug alternatives are presented, they are typically presented by therapeutic class, formulary status, and then alphabetically. The physician then determines which drug to prescribe at the point of care. The prescription is sent to the pharmacy through the physician's electronic health record (EHR) or electronic medical record (EMR) system, e.g., electronically, or carried by the patient, e.g., a printed paper prescription. If the patient is enrolled with a third-party for prescription benefits, the pharmacy submits a claim to the payer's system to determine the amount the patient must pay, the amount a health plan pays, and the amount the pharmacy receives for the prescribed drug.

Given the complexity of determining costs for a particular patient and a particular drug, it is often difficult for patients and physicians to make sound economic choices without sacrificing efficacy of the desired therapeutic drug treatment. It is desirable to have a system that provides appropriate cost and efficacy information to patients and prescribers, e.g., physicians, nurse practitioners, pharmacists, dentists, or other providers, to improve the prescription care and outcomes ultimately provided to the patient. It is further desirable to enhance F&B guidance to include such cost information and to help prescribers identify drug alternatives that are cost effective, clinically appropriate, and are administered by an appropriate route (e.g., orally or injected) and form (e.g., tablet or inhaler).

### BRIEF DESCRIPTION

One aspect of the methods described herein includes a method of processing drug alternatives for a selected drug. The method includes receiving a selection of the selected drug and generating a drug alternatives list from source data, the drug alternative list including a plurality of drug alternatives. The method includes gaining access to a price model and at least one price data source. The method includes determining respective prices for the plurality of drug alternatives according to the price model and the at least one price data source and ranking the plurality of drug alternatives in the drug alternatives list according to respective ordinality and respective prices. The method includes suppressing ones of the plurality of drug alternatives lacking a desired clinical outcome from the drug alternatives list. The method includes filtering resultant drug alternatives by at least one parameter. The method includes transmitting the drug alternatives list to a user computing system for display.

Another aspect of the methods described herein includes a method of approximating cost of a plurality of drug alternatives. The method includes receiving source data for the plurality of drug alternatives and importing corresponding price data for the plurality of drug alternatives. The method includes applying a respective randomly determined distortion to the corresponding price data for the plurality of drug alternatives to yield distorted price data. The method includes gaining access to a price model. The method includes generating formulary and benefit information according to the price model and the distorted price data.

### BRIEF DESCRIPTION OF THE DRAWINGS

FIG. 1 is an illustration of an example of a typical prescribing physician's view of F&B information;

FIG. 2 is an illustration of an example of an improved physician's view of F&B information;

FIG. 3A is an illustration of an example of a drug alternatives list sorted by formulary status and alphabetically;

FIG. 3B is an illustration of another example of a drug alternatives list sorted by formulary status, drug price, and alphabetically;

FIG. 3C is an illustration of another example of a drug alternatives list filtered by clinical efficacy and then sorted by formulary status, drug, price, and alphabetically;

FIG. 4 is a block diagram of one embodiment of a system in which drug alternatives are processed for a selected drug;

FIG. 5 is an illustration of an example of one embodiment of a software interface for a payer to configure a drug alternatives list;

FIG. 6 is an illustration of another example of the software interface shown in FIG. 5;

FIG. 7 is an illustration of an example of one embodiment of a software interface for a payer to view improved F&B data;

FIG. 8 is an illustration of an example of one embodiment of a software interface for a payer to view real-time prescription benefit check (RTPBC) data;

FIG. 9 is an illustration of an example of one embodiment of a software interface for a payer to opt in or out of a drug grouping and/or review drug groupings;

FIG. 10 is an illustration of an example of the software interface shown in FIG. 9 for selecting and reviewing a drug route or form filter;

FIG. 11 is an illustration of an example of one embodiment of a software interface for a payer to combine a formulary with a corresponding pricing model;

FIG. 12 is an illustration of an example of the software interface shown in FIG. 11 for ensuring data consistency among inbound data sources;

FIG. 13 is an illustration of an example of one embodiment of a software interface for a payer to create a pricing model;

FIG. 14 is an illustration of an example of the software interface shown in FIG. 13 for modifying a pricing model;

FIG. 15 is an illustration of an example of one embodiment of a software interface for creating a pricing source to be used in the pricing models shown in FIG. 13 and FIG. 14;

FIG. 16 is an illustration of an example of the software interface shown in FIG. 15 for further configuring a pricing source; and

FIG. 17 is a flow diagram of one example of a software implemented method of processing drug alternatives for a drug.

### DETAILED DESCRIPTION

Embodiments of the systems and methods described herein enable payers and providers to better manage drug and medical device costs for patients, improve patient satisfaction, maintain compliance with federal, state, and local laws, rules, and regulations, and better understand preferred drugs, medical devices, and alternatives. Embodiments may include software and software-implemented methods to provide improved formulary and benefit (F&B) information to payers and providers, including group insurance information, copay amounts in dollars and/or percentages (and including 30-day and 90-day pricing), alternative drug options, messaging to providers and patients through electronic medical records (EMR) or electronic health records (EHR), and real-time prescription benefit check (RTPBC) or other medical benefit check, such as for medical devices, at the point of care. Improved F&B information enables payers and providers to view drug alternatives by therapeutic class or therapeutic classes, formulary status, cost, and alphabetically and further enables filtering drugs by route, form, and clinical use. Route refers to the route of drug administration, such as, for example, inhalation, injection, mouth/throat, ophthalmic, or oral. Form refers to the physical form of the drug, such as, for example, liquid, nasal, intravenous, tablet, capsule, powder, or other solid. The improved F&B information may be further extended to patient portals, electronic prescribing (e-prescribing) portals, RTPBC portals, pharmacy system portals, and mobile portals.

In certain embodiments of the systems and methods described herein, payers may customize the drug alternatives list, for example, to suppress drugs with a better formulary status, but with higher cost than less-preferred and less expensive drug alternatives. Such a customized drug alternatives list may be further configured to protect rebate contracts. For example, such a rebate contract may require a certain drug Y be displayed whenever another drug X is presented. The customized drug alternatives list may be further configured to exclude branded drugs.

In certain embodiments of the systems and methods described herein, payers may submit messages to providers to convey certain information, such as, for example, an approximate price of a drug. In certain such embodiments, the software or software-implemented method includes a pricing algorithm that randomly distorts precise payer pricing when submitting messages with approximate pricing. Such distortion secures proprietary or confidential pharmacy network contracting and rebated drug costs from reverse-engineering while providing approximate drug cost information to physicians and their patients.

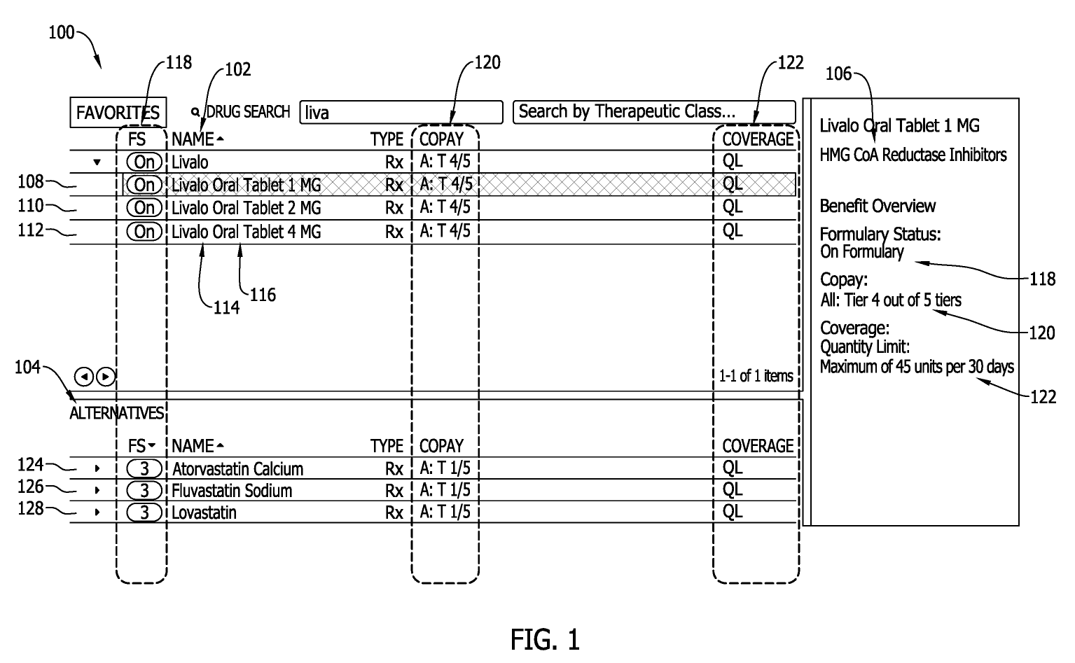

Typical F&B guidance enables providers to view which drugs are covered by a given patient's benefits, limited information on copays, e.g., copay tier, limited information on coverage limits, e.g., quantity limits, and formulary preferredness, e.g., formulary status. FIG. 1 illustrates an example of a typical prescribing physician's view 100 of F&B information for, for example, the cholesterol medication pitavastatin, otherwise referred to under its brand name Livalo®. When seeking to prescribe a drug 102 or an alternative 104 in its therapeutic class 106, the physician can view, for example, information for the drug 102 including its various dosages, i.e., drugs 108, 110, and 112. Alternatives 104 are generally other brand name drugs or generic formulations (or simply “generics”). The physician can also view a route 114 and a form 116 for each drug 102. The physician can also view limited F&B information that is generally not patient specific. For example, limited F&B information may include a formulary status 118. Formulary status 118 generally refers to whether a given drug is included on a payer's formulary list and, in certain cases, a preferred drug's preference level. For many drugs, generics and preferred brand names are “on-formulary, preferred” level one, or level two, etc., and less-preferred brand names are either “on-formulary, not preferred,” off/non-formulary, or non-reimbursable. A drug being “on-formulary, not preferred,” off/non-formulary, or non-reimbursable generally corresponds to an increasingly higher cost under the enrolled benefit for the payer. Likewise, the physician can typically view a copay tier 120. A lower copay tier 120 generally corresponds to a lower cost for the patient, while a higher copay tier 120 corresponds to a higher cost. Copay tiers typically vary among payers, e.g., PBMs and group insurance plans. In other words, a given drug may be tier 1 with one payer, and tier 3 for another. The physician can typically view any coverage limits 122 that may exist for a given drug. For example, a given drug may have a quantity limit for a specified period of time, such as, for example, 45 tablets every 30 days.

Given the limited information illustrated in FIG. 1, the physician decides which of drug 102 and alternatives 124, 126, and 128 to prescribe for a particular patient. In the case of prescribing such a cholesterol medication, the physician may first consider formulary status 118 to determine which drugs are on formulary (drugs 108, 110, 112) and which are more preferred, e.g., alternatives 124, 126, and 128. The physician may also consider copay tier 120, which identifies alternatives 124, 126, and 128 as potentially lower cost than on-formulary drugs 108, 110, and 112. Notably, the physician does not have access to specific drug costs for a particular patient and may not know the relative potency of each drug alternative. Consequently, the physician may prescribe a more costly drug and likely must reference compendia data, consult a pharmacist, or some other information source to determine the efficacy of a given drug alternative.

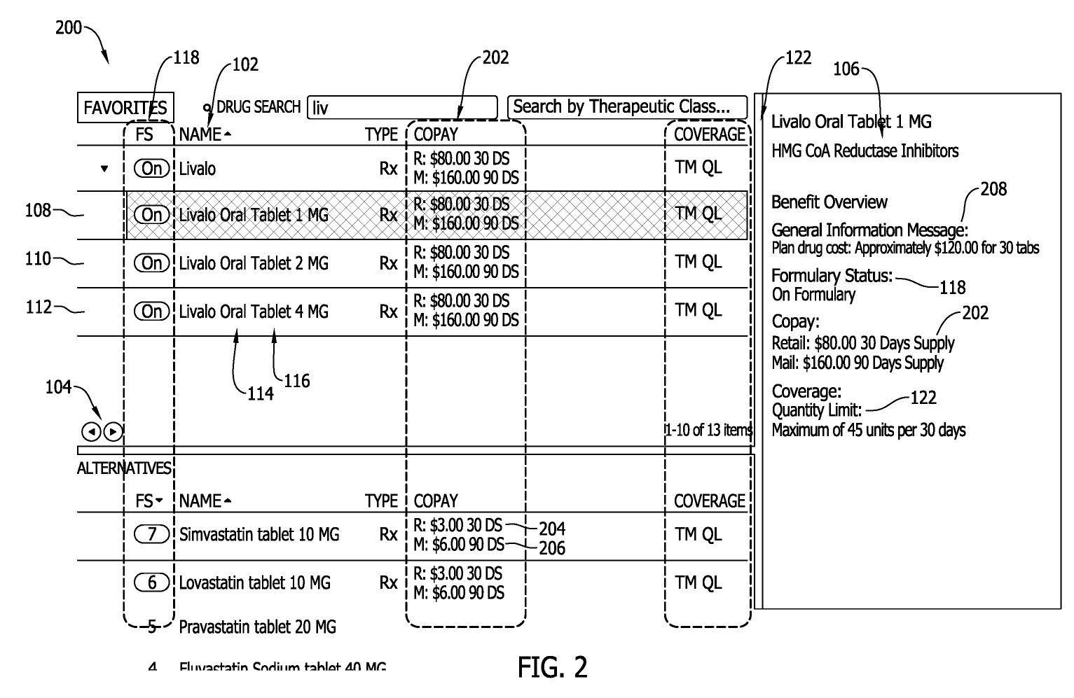

FIG. 2 illustrates an example of an improved physician's view 200 of F&B information according to an embodiment of the software or software-implemented methods described herein. The improved view 200 includes, for each drug 102 and drug alternatives 104, formulary status 118 and coverage limits 122. Rather than providing the copay tier 120 information illustrated in FIG. 1, the improved F&B information includes actual copay information 202 expressed in, for example, dollars. Alternatively, copay information 202 may be expressed as a percentage. As shown in FIG. 2, copay information 202 includes amounts for a “retail” 30 day supply 204 and a “mail order” 90 day supply 206. Further, the improved F&B information includes a message 208 provided by the payer to the physician through an EMR for that particular patient. Message 208 may, in certain embodiments, convey the actual cost of the drug under the pharmacy benefits for that particular patient. Given the improved F&B information illustrated in FIG. 2, the physician can decide which of drug 102 and drug alternatives 124 and 128 is cost effective to prescribe for the patient.

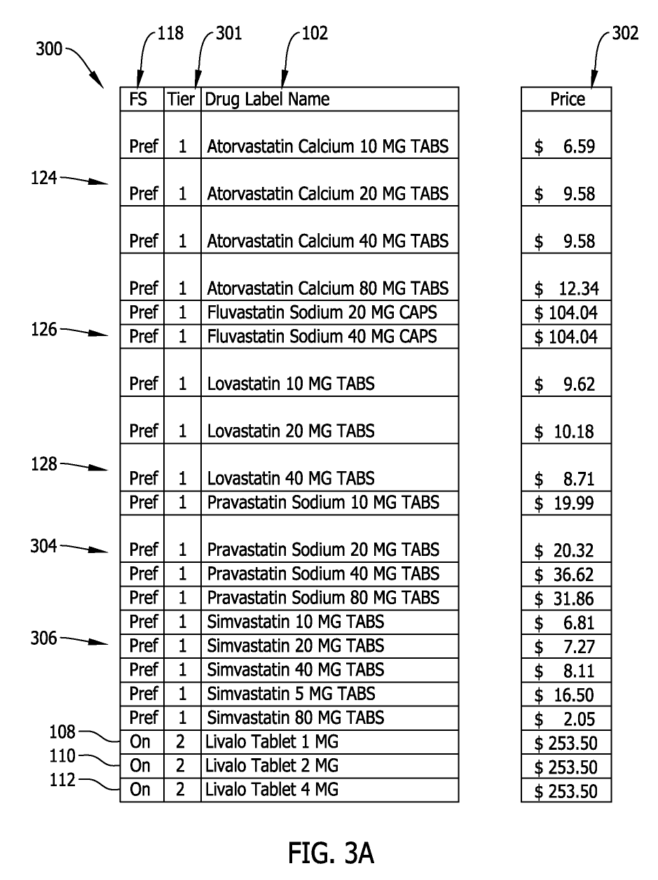

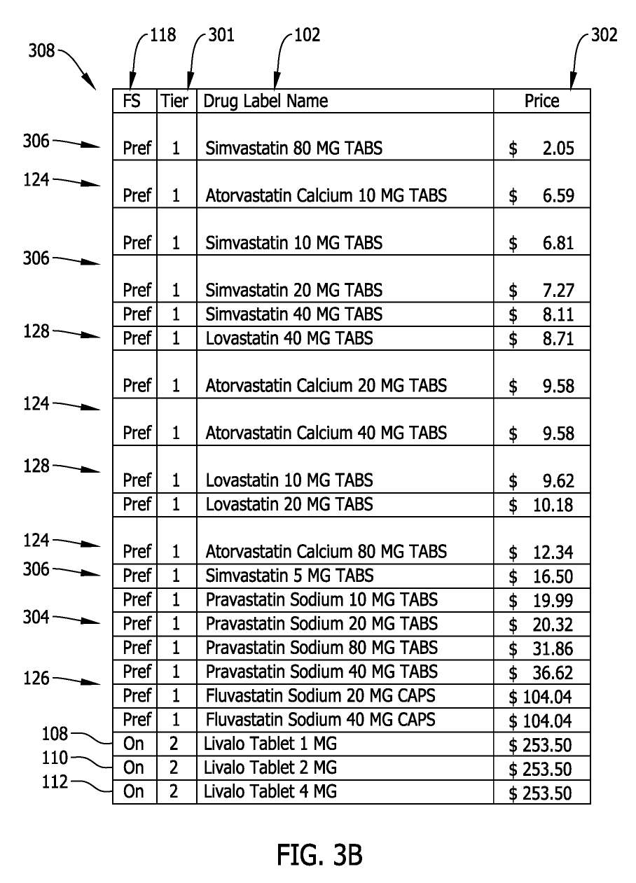

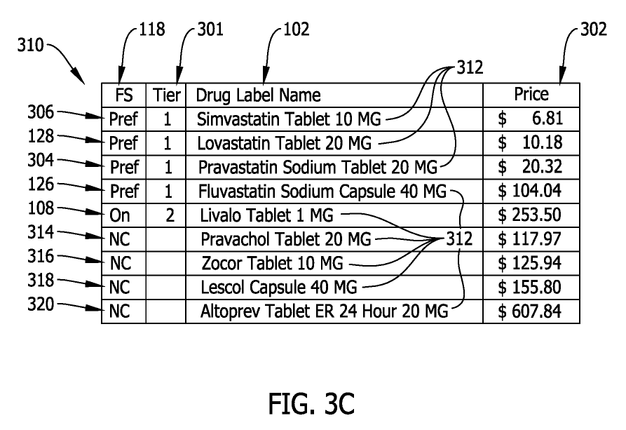

FIG. 3A illustrates an example of a drug alternatives list 300 sorted by formulary status 118 and alphabetically. In the example of FIG. 3A, an ordinal tier 301 corresponds with formulary status; however, in other instances, ordinal tier 301 may not correspond with formulary status. Notably, drug alternatives list 300 does not include respective price data 302 for the drugs, which are shown separately in FIG. 3A for reference only. Accordingly, certain drug alternatives have a preferred formulary status 118 within a given copay tier 120, and appear nearer the top of drug alternatives list 300 when sorted alphabetically. Conversely, certain drug alternatives, such as pravastatin sodium 304 and Simvastatin 306 appear nearer the bottom of drug alternatives list 300 when sorted alphabetically. Given the lack of price data 302 in drug alternatives list 300, the most cost-effective drug alternatives are not necessarily ranked high in the list, resulting potentially in extra cost for both the patient and the payer.

FIG. 3B illustrates an example of a drug alternatives list 308 based on F&B information according to an embodiment of the software or software-implemented methods described herein. Drug alternatives list 308 is sorted by formulary status 118, price data 302, and alphabetically. Given that price data 302 is incorporated into the F&B information on which drug alternatives list 308 is based, embodiments of the software or software-implemented methods described herein identify lowest cost drug alternatives. For example, in the example illustrated in FIG. 3B, certain Simvastatin dosages 306 rise in drug alternatives list 308, and fluvastatin sodium dosages fall in drug alternatives list 308. Although drugs 102 are sorted by formulary status and then price data 302, the physician still must determine which dosages of the various drug alternatives 108, 110, 112, 124, 126, 128, 304, and 306 are appropriate for the patient.

FIG. 3C illustrates another example of a drug alternatives list 310 based on F&B information according to another embodiment of the software or software-implemented methods described herein. Drug alternatives list 310 is filtered by at least one parameter, such as dosage, form, route, or may be grouped, categorized, or binned into subsets based on at least one parameter, such as dosage, form, route, or clinical expertise, or clinical data. For example, in the embodiment shown in FIG. 3C, drug alternatives list 310 is filtered by clinical efficacy, and then sorted by formulary status 118, price data 302, and alphabetically. Clinical efficacy is sometimes referred to as clinical determination, i.e., dosages 312 having approximately equal efficacy. Given the clinical determination, the physician can eliminate dosages 312 of drug alternatives that are inappropriate for the patient. Drug alternatives 314, 316, 318, and 320 would not be displayed to a physician, but are listed as potential trigger drugs. In the example of FIG. 3C, only on-formulary or preferred, level 1 or higher drugs are displayed to physicians, even though their respective formulary statuses 118 are not covered. In certain embodiments, only alternative drugs having a better formulary status 118 than a trigger drug are displayed in drug alternatives list 310.

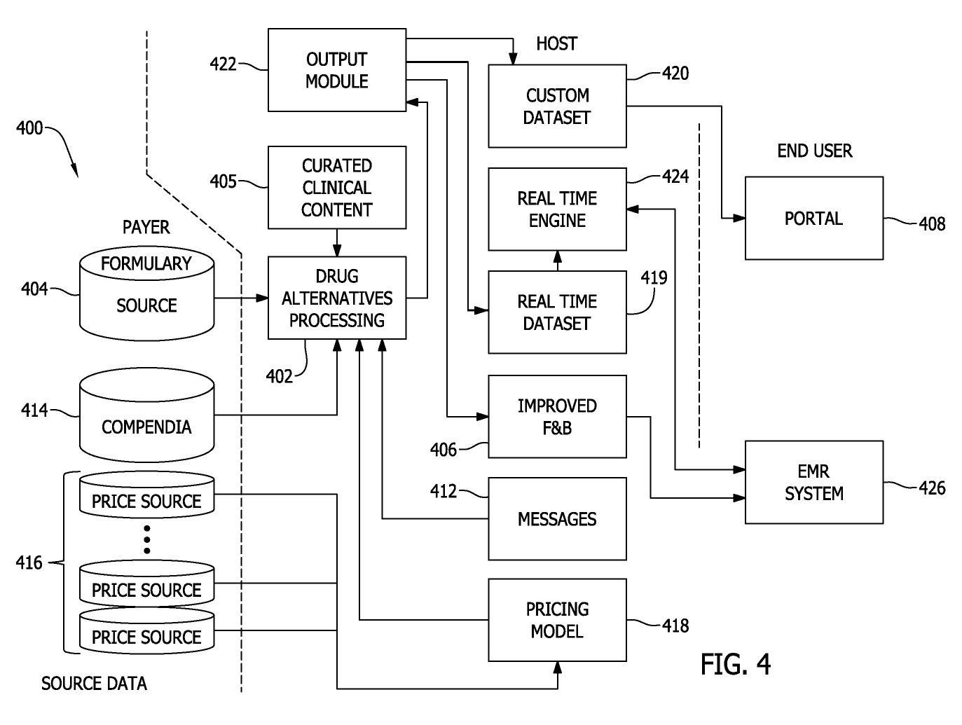

FIG. 4 is a block diagram of one embodiment of a software system 400 in which drug alternatives are processed for a selected drug. System 400 includes a drug alternative processing module 402 that modifies formulary data 404 to produce improved F&B data 406 and other real-time or custom data sets as described above with respect to FIGS. 2 and 3. In certain embodiments, drug alternative processing module 402 receives source data such as F&B data or curated clinical content 405 for a given payer and provides drug alternatives, developed directly from one or more other source data, to an output module 422. In alternative embodiments, drug alternative processing module 402 receives other formulary data from a provider, patient, payer, or other administrator or entity. Output module 422 formats the drug alternatives into certain formats such as improved F&B data 406, a real-time data set 419, and/or a custom data set 420.

Users, such as payers, patients, and/or providers gain access to improved F&B data 406, real-time data set 419, or custom data set 420 using a computing system and a portal 408. For providers, such a computing system may include a prescribing system that is typically part of an EHR or EMR system 426. In certain embodiments, a user may gain access to improved F&B data 406 using portal 408 to the prescribing system or other computing system for that particular user, such as a patient portal, an electronic prescribing (e-prescribing) portal, a RTPBC portal in combination with a real-time engine 424, a pharmacy system portal, or a mobile portal. Prescribing systems may further gain access, create, and modify an EMR. An EMR is a collection of medical data pertaining, for example, to a particular patient. EMRs are created, maintained, and modified using various EMR systems 426 that vary, for example, from provider to provider. The EMR is utilized, for example, by the provider to generate a prescription. The EMR system 426 may also incorporate one or more portions of improved F&B data 406. In certain embodiments, system 400 may display messages 412, attached to improved F&B data 406 by drug alternatives processing module 402, within EMR system 426 to convey additional information to the provider or patient, such as, for example, actual drug cost, medical history, or other considerations of which the provider or the patient should be aware.

Drug alternative processing module 402 utilizes various inputs, including formulary data 404 for the payer and compendia data 414. Drug alternative processing module 402 further utilizes data from price sources 416, or “price data.” Price data may include, for example, one or more of the payer's copay tier model, dispensing fees set by the payer, MACs, WACs, AWPs, national average drug acquisition costs (NADAC), rebate files, discounted cash pay prices, other pricings sources, and claim files. NADAC data includes data procured and aggregated directly from pharmacies on their actual costs. Rebate files include data setting out basic contract prices for pharmacies, and payers. Claim files include data representing actual pharmacy benefit claims.

Drug alternative processing module 402 determines what price information to include in improved F&B data 406 based on a pricing model 418 constructed using a software interface that enables a payer to manage the various price data from price sources 416. For example, pricing model 418 may include a hierarchical arrangement of the different data elements from price sources 416. When processing formulary data 404, drug alternative processing module 402 references pricing model 418 to determine which of price sources 416 should be used to incorporate into improved F&B data 406. For example, in one embodiment, a payer may define pricing model 418 to give preference to NADAC price data when available for a given drug alternative.

Generally, EMR system 426 is a software-implemented system executing on a computing system of the provider. In at least some embodiments, EMR system 426 includes a prescribing system software module integrated into EHR system 426. In alternative embodiments, for some providers, the prescribing system operates independently of their respective EHR system. A provider interacts with the computing system to gain access to and view, for example, improved F&B data 406 and real-time data set 419 to perform a RTPBC and further enable the provider to generate a prescription. EHR system 426 enables a patient or provider to view medical data, such as improved F&B data 406, and any messages 412 relevant to generating the prescriptions.

Generally, a host, or host system, operates a software-independent system including drug alternatives processing module 402. In certain embodiments, the payer hosts the system and users, such as providers, patients, or even users associated with the payer gain access to improved F&B data 406, real-time data set 419, and/or custom data set 420 locally or remotely using portal 408 and a network connection using, for example, the Internet. The payer provides drug alternatives processing module 402 with access to various data including, for example, formulary data 404, compendia data 414, and price data from price sources 416 that may be stored locally or remotely on one or more mass storage memory devices. For example, formulary data 404, compendia data 414, curated clinical content 405, and prices sources 416 may be stored locally with the host system, or may be coupled remotely to the host system over a network connection. In certain embodiments, drug alternatives processing module 402 and one or more of pricing module 418, output module 422, and real-time engine 424 are hosted remotely for the payer by a third-party entity. Drug alternatives processing module 402 also utilizes pricing model 418, which includes a memory structure within which rules and algorithms for processing pricing data are defined.

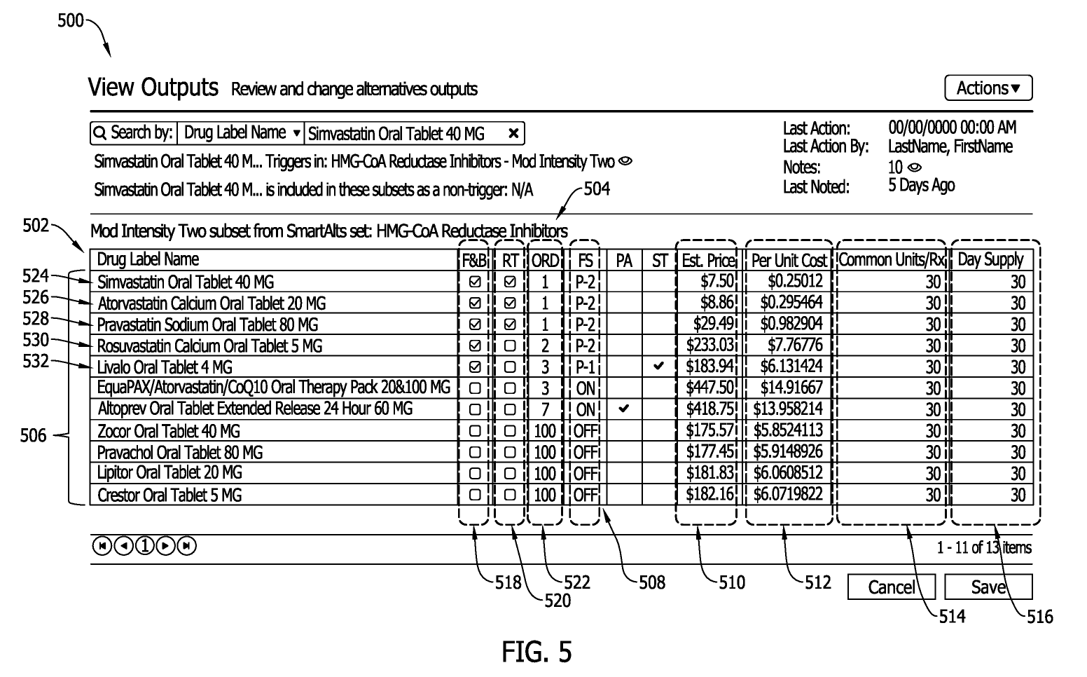

FIG. 5 is an illustration of an example of one embodiment of a software interface 500 for a payer to configure a drug alternatives list 502. In the example of FIG. 5, software interface 500 enables the payer to configure drug alternatives list 502 for the therapeutic class 504 of drugs of HMG-CoA Reductase Inhibitors. Software interface 500 presents (recognizing that only on-formulary or preferred, level 1 or higher level preferred drugs are displayed to physicians as drug alternatives) the various drug alternatives 506 in the therapeutic class 504 or classes, including a formulary status 508, estimated price 510, a per unit cost 512, a baseline units per prescription (units/Rx) 514, and a number of days of supply 516 for baseline units/Rx 514. Software interface 500 further enables the payer to configure each of drug alternatives 506 to be included or excluded in the improved F&B data 518, and to be included or excluded in RTPBC data 520. In certain embodiments, only drugs selected for improved F&B data 518 or for RTPBC data 520 are displayed to a physician in their improved F&B portal or RTPBC portal. Software interface 500 further enables the payer to view the ultimate ranking 522, or ordering, of drug alternatives 506 when a provider gains access to the improved F&B data. For example, in the example shown in FIG. 5, for therapeutic class 504, drug alternatives 524, 526, 528, 530, and 532 are included in the improved F&B data 518. Further, in the example shown in FIG. 5, drug alternatives 524, 526, and 528 are included in the RTPBC data 520.

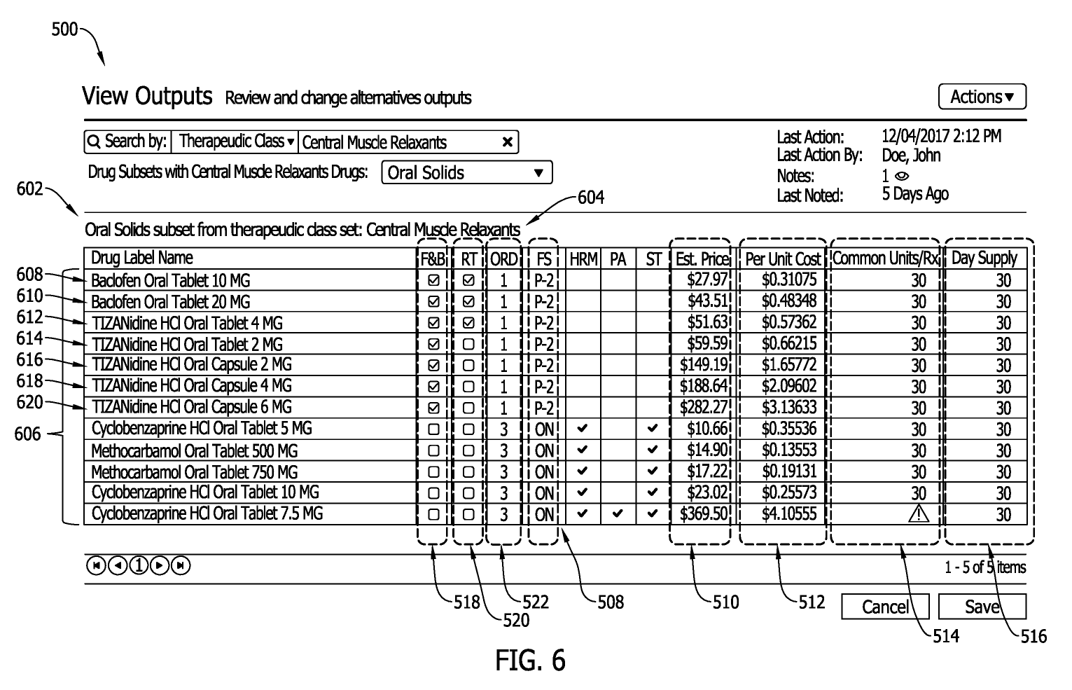

FIG. 6 is an illustration of another example of the software interface 500 shown in FIG. 5. In the example shown in FIG. 6, the payer configures a drug alternatives list 602 for a therapeutic class 604 of central muscle relaxants. Software interface 500 presents various drug alternatives 606 in the therapeutic class 604, including formulary status 508, estimated price 510, per unit cost 512, baseline units/Rx 514, and number of days of supply 516 (all shown in FIG. 5). Software interface 500 further enables payer to configure each of drug alternatives 606 to be included or excluded in the improved F&B data 518, and to be included or excluded in RTPBC data 520. Software interface 500 further enables the payer to view the ranking 522, or ordering, of drug alternatives 606 when a provider gains access to the improved F&B data through a prescribing system. For example, in the example shown in FIG. 6, for therapeutic class 604, drug alternatives 608, 610, 612, 614, 616, 618, and 620 are included in the improved F&B data 518. Further, in the example shown in FIG. 6, drug alternatives 608, 610, and 612 are included in the RTPBC data 520.

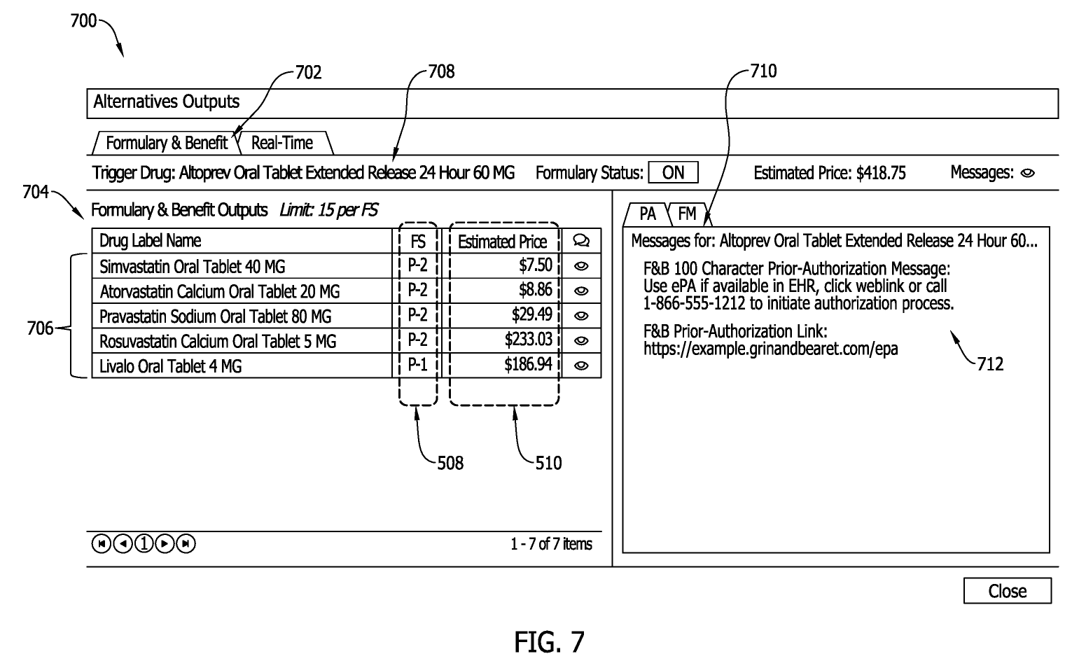

FIG. 7 is an illustration of an example of one embodiment of a software interface 700 for a payer to view improved F&B data. The improved F&B data 702 includes a drug alternatives list 704 that includes formulary status 508 and estimated price 510 for each of drug alternatives 706. In the example shown in FIG. 7, drug alternatives list 704 is presented to a provider when the provider desires to prescribe a trigger drug 708, e.g., Altoprev Oral Tablet Extended Release 24 Hour 60 MG based on the configuration from FIG. 5. Software interface 700 enables the payer to configure drug alternative list 704 to include drug alternatives 706 arranged according to their respective formulary status 508 and estimated price 510. Further F&B information may also be presented as one or more messages 710. Messages 710 may include, for example, prior authorization information 712 or, alternatively, actual cost information for the patient.

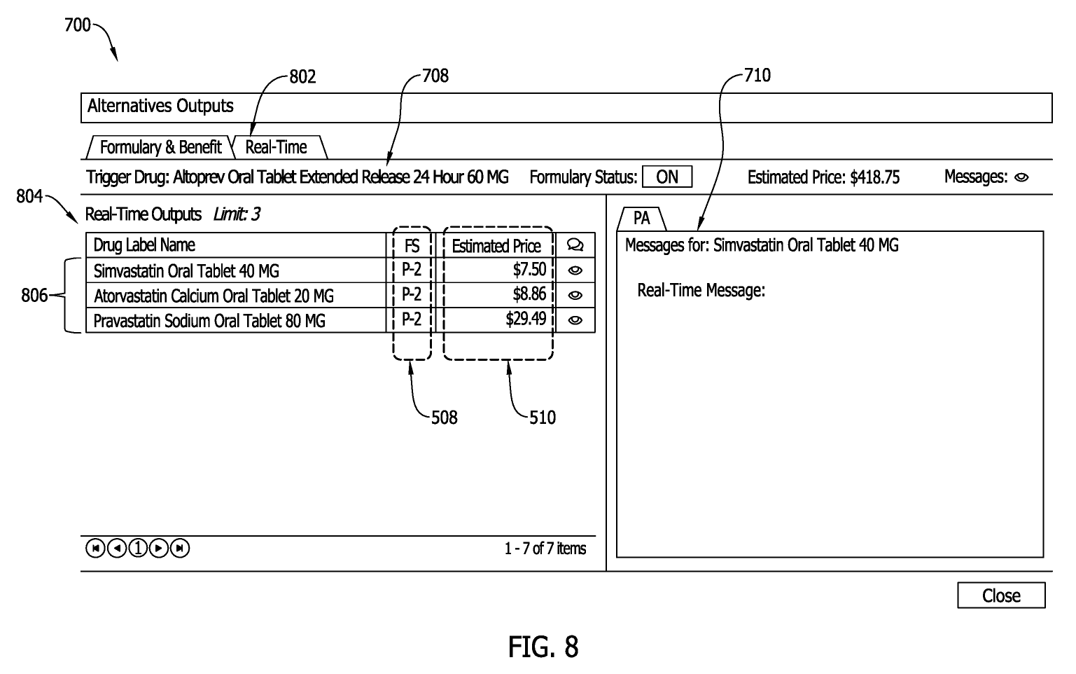

FIG. 8 is an illustration of an example of one embodiment of software interface 700 shown in FIG. 7. Software interface 700 enables the payer to view RTPBC data 802. RTPBC data 802 includes a drug alternatives list 804 that includes formulary status 508 and estimated price 510 for each of drug alternatives 806. In the example shown in FIG. 8, drug alternatives list 804 is presented to a provider when the provider desires to prescribe trigger drug 708 based on the configuration from FIG. 6. Software interface 700 further enables display the payer's limits on drug alternatives list 804 to certain drugs for RTPBC, which, in this case, are three of the five drug alternatives 706 shown in the improved F&B data illustrated in FIG. 7.

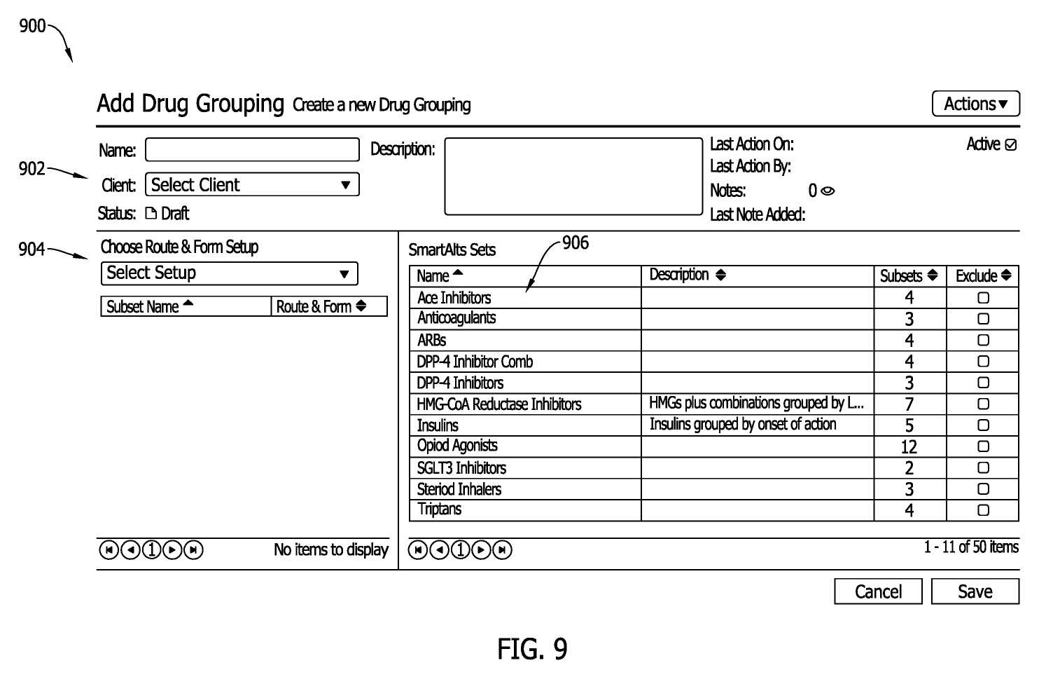

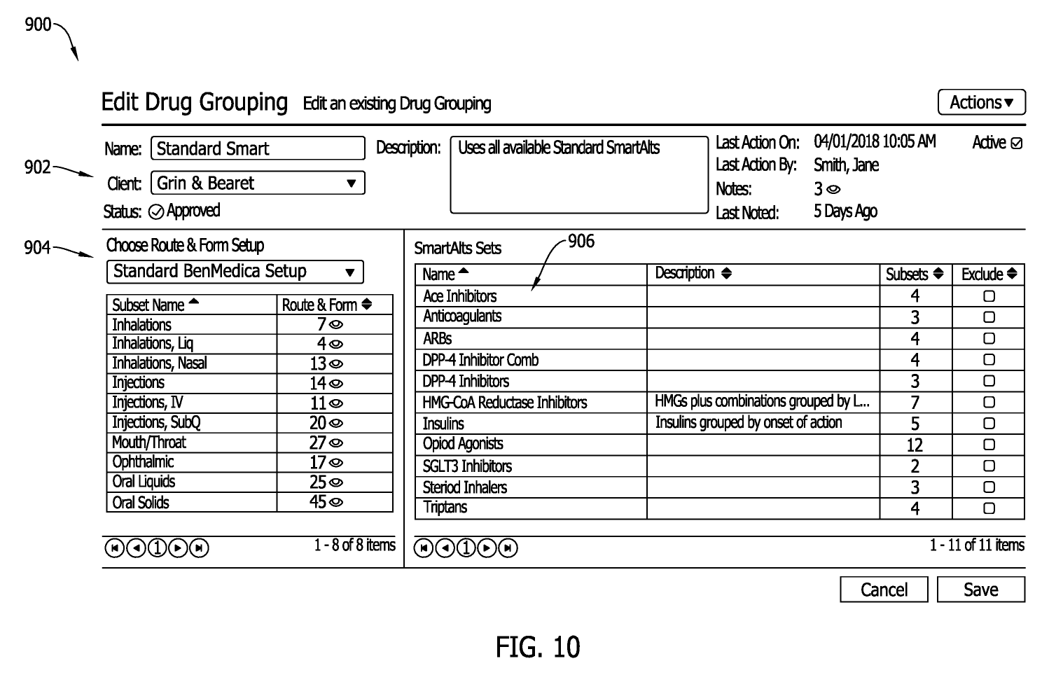

FIG. 9 is an illustration of an example of one embodiment of a software interface 900 for a payer to opt in or out of, or create, a custom drug grouping 902, and to review drug groupings 902. Software interface 900 enables drug grouping 902 to be defined according to, for example, one or more route and form filter 904, and/or enable or disable one or more managed therapeutic classes 906. FIG. 10 is an illustration of software interface 900 shown in FIG. 9 for selecting and reviewing a drug route and form filter.

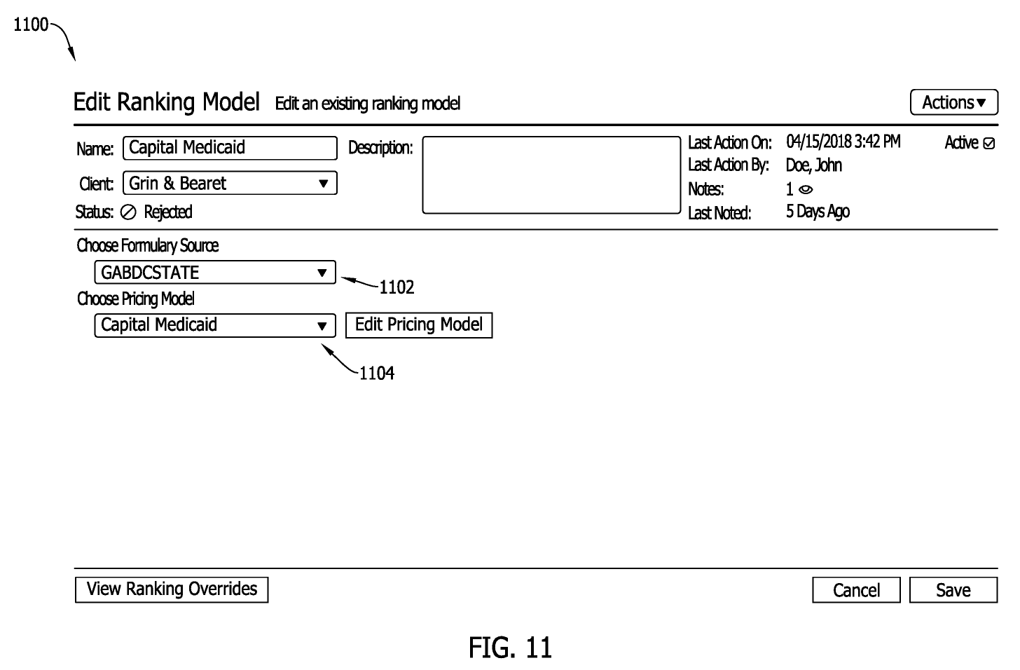

FIG. 11 is an illustration of an example of one embodiment of a software interface 1100 for a payer to combine a formulary with its corresponding pricing model. Software interface 1100 enables a payer to select and view a particular formulary source 1102, e.g., a specific set of F&B data. The formulary source 1102 is the baseline on which, for example, drug alternatives processing module 402 builds improved F&B data that, for example, includes pricing data from price sources 416 (shown in FIG. 4). Software interface 1100 further enables the payer to select a pricing model 1104, such as pricing model 418 (shown in FIG. 4). Pricing model 1104 is then used, for example, by drug alternatives processing module 402 to build improved F&B data from formulary source 1102.

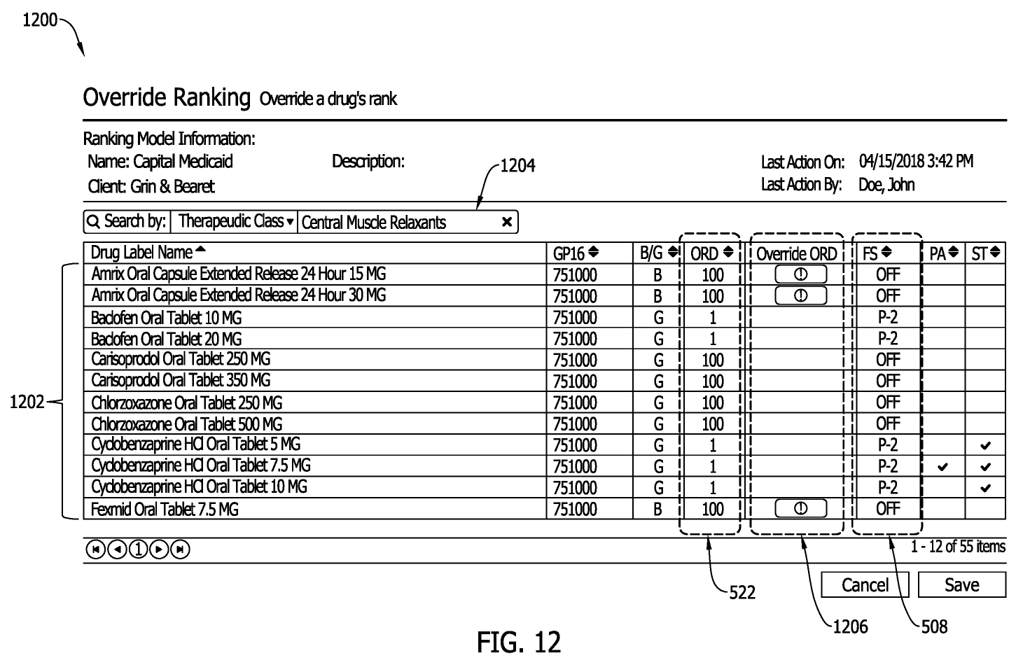

FIG. 12 is an illustration of an example of software interface 1100 shown in FIG. 11 for ensuring data consistency among inbound data sources, including, for example, configuring rankings of drug alternatives 1202 for a given therapeutic class 1204. Software interface 1100 displays each of drug alternatives 1202 with their respective formulary status 508 and rank 522. Software interface 1100 further enables the payer to provide a rank override 1206.

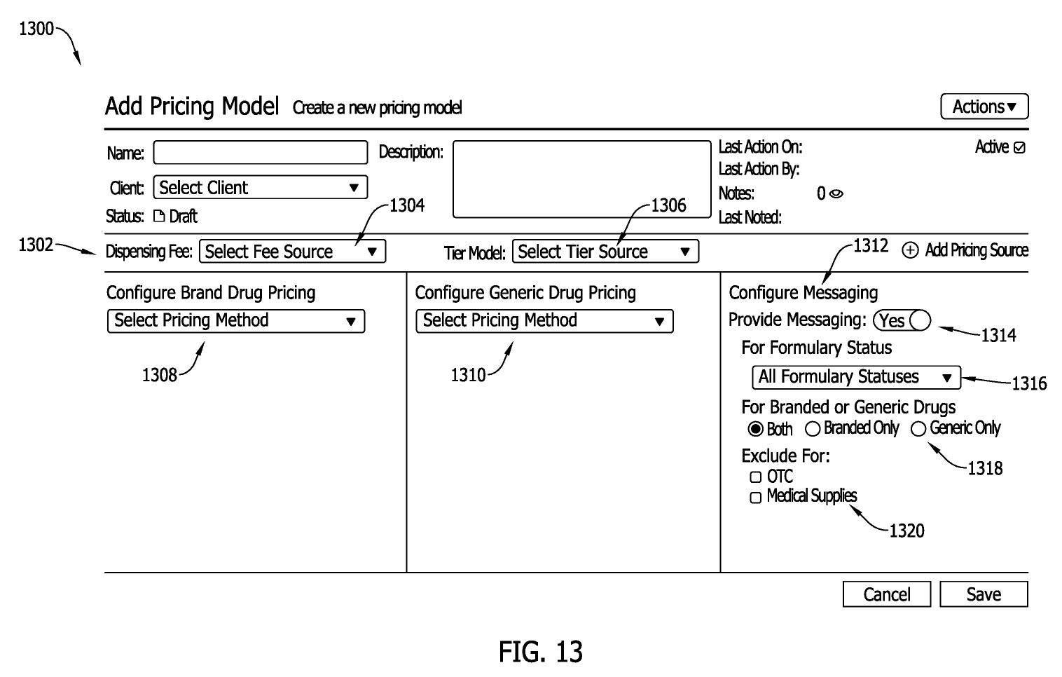

FIG. 13 is an illustration of an example of one embodiment of a software interface 1300 for a payer to create a pricing model 1302 that applies generally for all drugs within a formulary. Software interface 1300 enables the payer to select various data sources to incorporate into pricing model 1302. For example, software interface 1300 enables the payer to select a data source for dispensing fees 1304, or enter a custom amount, and a formulary tier model 1306. Software interface 1300 further enables the payer to select a price data source, such as one of the various price data from price sources 416 shown in FIG. 4, to be used for brand name drugs 1308 and for generic drugs 1310. Software interface 1300 further enables the payer to configure drug cost messaging 1312 for the drugs generally and, particularly, to enable or disable messaging 1314 entirely. Software interface 1300 enables the payer to further enable or disable drug cost messaging for particular formulary statuses 1316, for brand name or generic drugs 1318, and for various other types of medical or pharmaceutical goods 1320, such as, for example, medical supplies or over-the-counter medications.

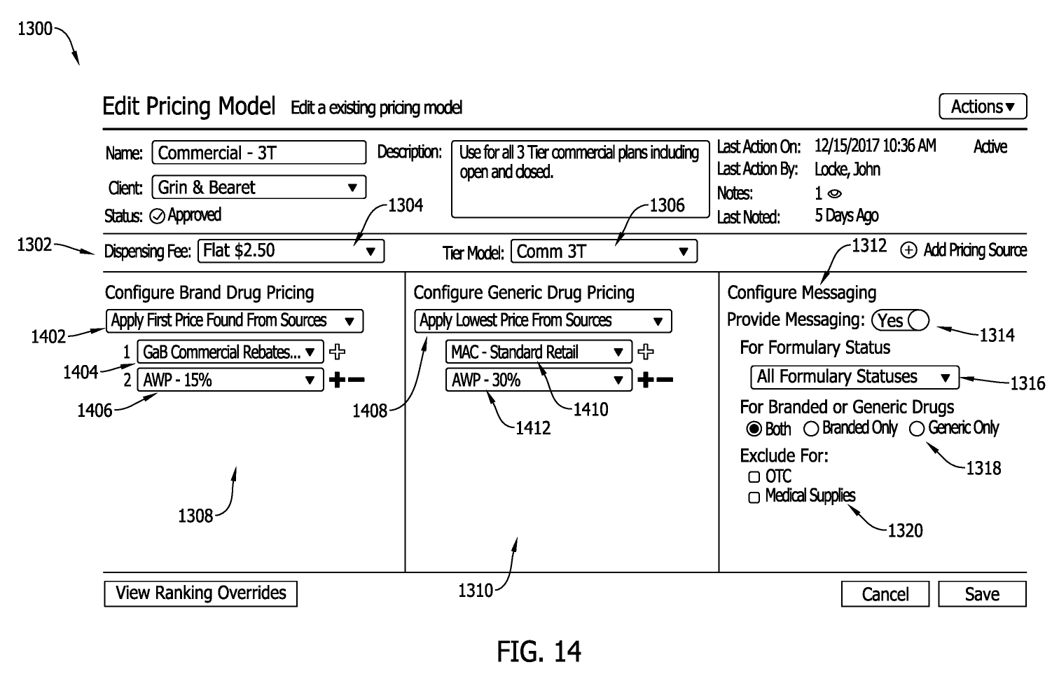

FIG. 14 is an illustration of software interface 1300 shown in FIG. 13 for modifying pricing model 1302. As described above with respect to FIG. 13, software interface 1300 enables the payer to configure price data sources for brand name drugs 1308 and for generic drugs 1310. More specifically, for example, software interface 1300 enables the payer to set a brand name price rule 1402 that dictates how multiple price data sources 1404 and 1406 should be utilized for determining price data for brand name drugs that is incorporated into improved F&B data. In the example shown in FIG. 14, brand name price rule 1402 dictates that a first-found price is used in pricing model 1302 for brand name drugs. Price data sources 1404 and 1406 are, for example, rebates files and AWP with a 15% discount, respectively. Similarly, software interface 1300 enables the payer to set a generic price rule 1408 that dictates how multiple price data sources 1410 and 1412 should be utilized for determining price data for generic drugs that is incorporated into improved F&B data. Generic price rule 1408 may include one or more of a lowest price, a highest price, a maximum allowable cost, an average price, or a weighted average, for example, from among price data sources. In the example shown in FIG. 14, generic price rule 1408 dictates that a lowest price from among price data sources 1410 and 1412 is used in pricing model 1302 for generic drugs. Price data sources 1410 and 1412 are, for example, MAC and AWP with a 30% discount, respectively.

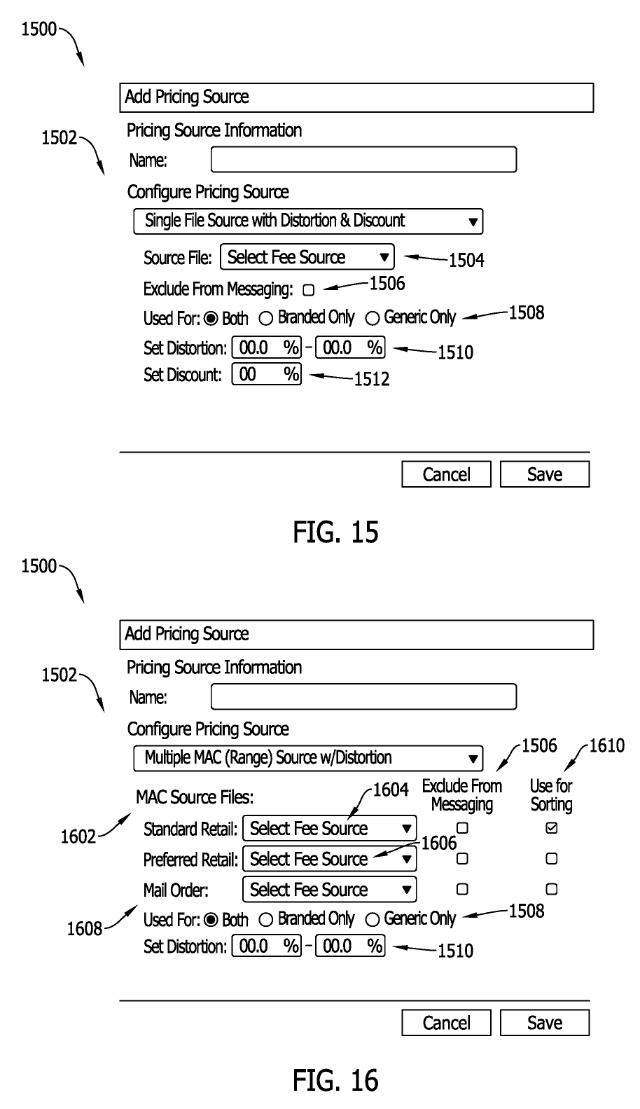

FIG. 15 is an illustration of an example of one embodiment of a software interface 1500 for creating a pricing source 1502 to be used in pricing model 1302 shown in FIG. 13 and FIG. 14. Software interface 1500 enables the payer to construct pricing source 1502 to utilize selected price data 1504. In certain embodiments, price data 1504 may include a single data file or, alternatively, may be a compilation of multiple data files. Software interface 1500 further enables the payer to include or exclude 1506 the content of pricing source 1502 from messaging that attaches to EHRs and may be distributed to providers and patients. The payer may, for example, decide to exclude 1506 the content of pricing source 1502 from messaging to protect proprietary information or confidential agreements with pharmacies or wholesalers. Software interface 1500 further enables pricing source 1502 to be configured 1508 for brand name drugs, generic drugs, or both. Software interface 1500 further enables pricing source 1502 to apply a random distortion 1510 to the content of pricing source 1502 to further protect pricing the content of pricing source 1502 from reverse engineering by providers, patients, payers, or other third parties. Software interface 1500 further enables pricing source 1502 to apply a discount 1512 to price data 1504. In certain embodiments, software interface 1500 may include an interface for receiving a distortion range input from a user, such as a payer.

FIG. 16 is an illustration of software interface 1500 shown in FIG. 15 for further configuring pricing source 1502. In the example shown in FIG. 16, pricing source 1502 is configured to utilize multiple sources of price data 1504, such as, for example multiple MAC sources 1602. Each of MAC sources 1602 may be designated for different pharmacy options, such as, for example, a standard retail pharmacy 1604, a preferred retail pharmacy 1606, and a mail order pharmacy 1608. As shown in FIG. 15, software interface 1500 also enables the payer to exclude or include 1506 content of MAC sources 1602 from messaging. Software interface 1500 enables the payer to identify which of MAC sources 1602 is to be utilized for ranking 1610, or sorting.

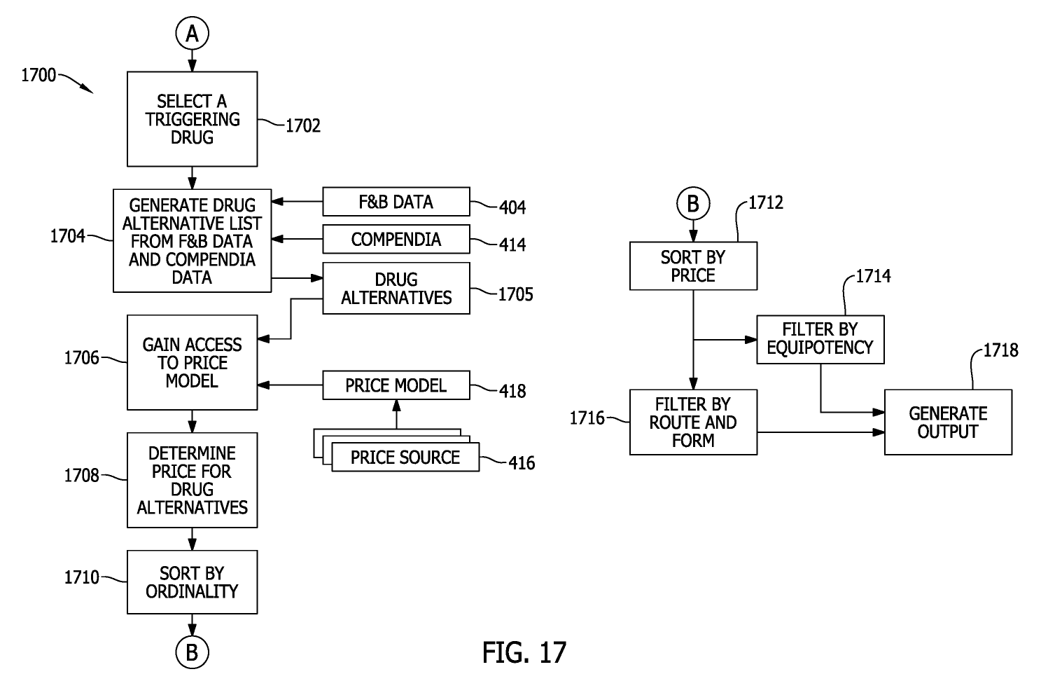

FIG. 17 is a flow diagram of one example of a software implemented method 1700 of processing drug alternatives for a selected drug. Method 1700 typically begins when a provider selects 1702 a particular drug or, in certain embodiments, a therapeutic class of drugs to be prescribed. In at least some embodiments, a single drug must be selected to determine drug alternatives, and each drug in a therapeutic class has alternatives. The user may make this selection using a computing system such as, for example, EMR system 426 communicatively coupled to a host system over, for example, a communication network using portal 408. Given the selection, the host system, using an embodiment of the software or software-implemented methods described herein, generates 1704 a drug alternatives list 1705. The host system utilizes, for example, drug alternatives processing module 402 (shown in FIG. 4) to generate a drug alternatives list 1705 from source data or augment existing formulary data 404 to generate drug alternatives list 1705 to incorporate price data from price sources 416. First, the host system gains access 1706 to a price model 418 and one or more price sources 416. Based on the rules and algorithms defined within price model 418, the host system determines 1708 prices for each of the drug alternatives and groups by formulary status to be included in the drug alternatives list, thereby incorporating one portion of improved F&B data 406. In certain embodiments, only on-formulary or preferred, level 1 or higher drugs are displayed to physicians. All other drugs in such embodiments are not included as drug alternatives, although they are normally assigned as drug alternatives by the system.

The host system then sorts 1710 the drug alternatives in the improved F&B data by ordinality to identify which drug alternatives are on-formulary or better preferred level. To sort by ordinality is to process to group certain drugs with the same level of preferredness where some groups are more preferred (or ranked higher) than other drugs with less preferred “ordinality.” Ordinality may be determined, for example, by formulary status or by plan tier assigned to a drug by a payer. The host system further sorts 1712 by price to identify, for the provider and the patient, which of the drug alternatives is the most cost-effective. The host system may further filter 1714 by clinical determination, or similar efficacy, filter 1716 by route and form, or both to further aid the provider in identifying both an efficacious and cost-effective drug among the drug alternatives. This data is exported to output module 422, which formats the data to generate output data 1718 such as improved F&B data 406, real time data 419, or custom data set 420 for viewing by a user computing system, such as the EMR system 426 such that the user then is able to view a list of drug alternatives for the patient based on a more complete data set than is otherwise available with the original formulary data 404 or compendia data 414. Alternatively, the user computing system may include portal 408 for viewing, configuring, or modifying improved F&B data 406, real time data 419, or custom data set 420.

The methods and systems described herein may be implemented using computer programming or engineering techniques including computer software, firmware, hardware or any combination or subset thereof, wherein the technical effect may include at least one of: (a) reducing drug costs for payers; (b) increasing use of mail and specialty orders; (c) improving patient adherence to preferred drugs; (d) reducing administrative costs; (e) improving awareness of therapeutic drug treatment options; (f) improving awareness of drug-specific guidance and alerts; (g) improving awareness of coverage restrictions; (h) reducing delays at pharmacies due to cost and coverage issues; (i) reducing out-of-pocket cost to patients; and (j) improving patient adherence to therapeutic drug treatment for chronic conditions.

In the foregoing specification and the claims that follow, a number of terms are referenced that have the following meanings.

As used herein, an element or step recited in the singular and preceded with the word “a” or “an” should be understood as not excluding plural elements or steps, unless such exclusion is explicitly recited. Furthermore, references to “example implementation” or “one implementation” of the present disclosure are not intended to be interpreted as excluding the existence of additional implementations that also incorporate the recited features.

“Optional” or “optionally” means that the subsequently described event or circumstance may or may not occur, and that the description includes instances where the event occurs and instances where it does not.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here, and throughout the specification and claims, range limitations may be combined or interchanged. Such ranges are identified and include all the sub-ranges contained therein unless context or language indicates otherwise.

Some embodiments involve the use of one or more electronic processing or computing devices. As used herein, the terms “processor” and “computer” and related terms, e.g., “processing device,” “computing device,” and “controller” are not limited to just those integrated circuits referred to in the art as a computer, but broadly refers to a processor, a processing device, a controller, a general purpose central processing unit (CPU), a graphics processing unit (GPU), a microcontroller, a microcomputer, a programmable logic controller (PLC), a reduced instruction set computer (RISC) processor, a field programmable gate array (FPGA), a digital signal processing (DSP) device, an application specific integrated circuit (ASIC), and other programmable circuits or processing devices capable of executing the functions described herein, and these terms are used interchangeably herein. The above examples are exemplary only, and thus are not intended to limit in any way the definition or meaning of the terms processor, processing device, and related terms.

In the embodiments described herein, memory may include, but is not limited to, a non-transitory computer-readable medium, such as flash memory, a random access memory (RAM), read-only memory (ROM), erasable programmable read-only memory (EPROM), electrically erasable programmable read-only memory (EEPROM), and non-volatile RAM (NVRAM). As used herein, the term “non-transitory computer-readable media” is intended to be representative of any tangible, computer-readable media, including, without limitation, non-transitory computer storage devices, including, without limitation, volatile and non-volatile media, and removable and non-removable media such as a firmware, physical and virtual storage, CD-ROMs, DVDs, and any other digital source such as a network or the Internet, as well as yet to be developed digital means, with the sole exception being a transitory, propagating signal. Alternatively, a floppy disk, a compact disc-read only memory (CD-ROM), a magneto-optical disk (MOD), a digital versatile disc (DVD), or any other computer-based device implemented in any method or technology for short-term and long-term storage of information, such as, computer-readable instructions, data structures, program modules and sub-modules, or other data may also be used. Therefore, the methods described herein may be encoded as executable instructions, e.g., “software” and “firmware,” embodied in a non-transitory computer-readable medium. Further, as used herein, the terms “software” and “firmware” are interchangeable, and include any computer program stored in memory for execution by personal computers, workstations, clients and servers. Such instructions, when executed by a processor, cause the processor to perform at least a portion of the methods described herein.

Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

The systems and methods described herein are not limited to the specific embodiments described herein, but rather, components of the systems and/or steps of the methods may be utilized independently and separately from other components and/or steps described herein.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to provide details on the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of processing drug alternatives for a selected drug, comprising:
receiving a selection of the selected drug;
generating a drug alternatives list from source data, the drug alternative list including a plurality of drug alternatives;
gaining access to a price model and at least one price data source;
determining respective prices for the plurality of drug alternatives according to the price model and the at least one price data source;
ranking the plurality of drug alternatives in the drug alternatives list according to respective ordinality and respective prices;
suppressing ones of the plurality of drug alternatives lacking a desired clinical outcome from the drug alternatives list;
filtering resultant drug alternatives by at least one parameter; and
transmitting the drug alternatives list to a user computing system for display.

2. The method of claim 1, wherein determining the respective prices for the plurality of drug alternatives comprises determining the respective prices according to actual pricing data for the patient under pharmacy benefits or cash pay for the patient.

3. The method of claim 1, wherein gaining access to at least one price data source comprises gaining access to a price data source selected from the group consisting of:
dispensing fees set by a payer,
maximum allowable cost (MAC),
wholesale acquisition cost (WAC),
average wholesale price (AWP),
national average drug acquisition costs (NADAC),
rebate files,
discounted cash pay price, and
claim files.

4. The method of claim 1 further comprising transmitting a message to a user computing system, the message including respective actual costs of the plurality of drug alternatives.

5. The method of claim 4, wherein the message includes respective coverage limits for the plurality of drug alternatives.

6. The method of claim 1, wherein transmitting the drug alternatives list to the user computing system comprises transmitting the drug alternatives list within an electronic medical record (EMR) system.

7. The method of claim 1, wherein determining the respective prices for the plurality of drug alternatives comprises determining respective per unit costs for the plurality of drug alternatives.

8. The method of claim 1 further comprising removing at least one of the plurality of drug alternatives from the drug alternatives list according to a configuration set by a payer.

9. The method of claim 1 further comprising re-ranking at least one of the plurality of drug alternatives in the drug alternatives list according to a configuration set by a payer.

10. The method of claim 1 further comprising removing at least one of the plurality of drug alternatives from the drug alternatives list based on similar efficacy with respect to the trigger drug.

11. A method of approximating cost of a plurality of drug alternatives, comprising:
receiving source data for the plurality of drug alternatives;
importing corresponding price data for the plurality of drug alternatives;
applying a respective randomly determined distortion to the corresponding price data for the plurality of drug alternatives to yield distorted price data;
gaining access to a price model; and
generating formulary and benefit information according to the price model and the distorted price data.

12. The method of claim 11, wherein receiving source data includes receiving formulary and benefit information comprising respective formulary status information and respective copay tier information for the plurality of drug alternatives.

13. The method of claim 11, wherein importing corresponding price data comprises importing respective price data for the plurality of drug alternatives from a price data source selected from the group consisting of:
dispensing fees set by a payer,
maximum allowable cost (MAC),
wholesale acquisition cost (WAC),
average wholesale price (AWP),
national average drug acquisition costs (NADAC),
rebate files,
discounted cash pay price, and
claim files.

14. The method of claim 11 further comprising configuring the price model to utilize a first price data source for brand name drugs among the plurality of drug alternatives.

15. The method of claim 14 further comprising configuring the price model to utilize a second price data source for generic drugs among the plurality of drug alternatives.

16. The method of claim 11, further comprising configuring the price model to apply a rule in utilizing a plurality of price data sources for the plurality of drug alternatives.

17. The method of claim 16, wherein the rule comprises applying a first price found in the plurality of price data sources organized in a priority order.

18. The method of claim 16, wherein the rule comprises applying one or more of a lowest price, a highest price, a maximum allowable cost, an average price, or a weighted average among the plurality of price data sources.

19. The method of claim 11 further comprising creating a new price data source based on at least one of a plurality of price data sources.

20. The method of claim 19 further comprising configuring the new price data source to apply a discount to the at least one of the plurality of price data sources.

21. The method of claim 11 further comprising receiving a distortion range input from a payer.
